(19) United States
(12) Patent Application Publication
(10) Pub. No.: US 2013/0053674 A1
(43) Pub. Date: Feb. 28, 2013

(54) ELECTROCARDIOGRAM MONITORING DEVICES

(71) Applicant: Monica Ann Volker, Lake Geneva, WI (US)

(72) Inventor: Monica Ann Volker, Lake Geneva, WI (US)

(21) Appl. No.: 13/582,299

(22) PCT Filed: Mar. 2, 2011
(86) PCT No.: PCT/US2011/026866

§ 371 Date: Nov. 5, 2012

Related U.S. Application Data
(60) Provisional application No. 61/310,185, filed on Mar. 3, 2010.

(51) Int. Cl. A61B 5/0408

(52) U.S. Cl. 600/389; 600/388

(57) ABSTRACT

The invention relates to devices and methods for monitoring cardiac function. Specifically, the invention relates to devices for secure placement of electrocardiogram (ECG) sensors (patches, electrodes) on the body of a subject.

This application claims priority to U.S. Provisional Ser. No. 61/310,185 filed Mar. 3, 2010, which is incorporated by reference in its entirety.

### FIELD OF THE INVENTION

The invention relates to devices and methods for monitoring cardiac function. Specifically, the invention relates to devices for secure placement of electrocardiogram (ECG) sensors (e.g., patches, electrodes) on the body of a subject.

### BACKGROUND OF THE INVENTION

Electrocardiography (commonly abbreviated as ECG, less commonly as EKG) is a transthoracic interpretation of the electrical activity of the heart over time, said activity captured and externally recorded by skin electrodes. ECG is a noninvasive recording produced by an electrocardiographic device. Electrical impulses in the heart originate in the sinoatrial node and travel through the intimate conducting system to the heart muscle. The impulses stimulate the myocardial muscle fibers to contract and thus induce systole. The electrical waves can be measured at electrodes placed at specific points on the skin. Electrodes on different sides of the heart measure the activity of different parts of the heart muscle. An ECG displays the voltage between pairs of these electrodes, and the muscle activity that they measure from different directions can be understood as vectors. This display indicates overall rhythm of the heart and weaknesses in different parts of the heart muscle. ECG is the preferred clinical method to measure and diagnose abnormal rhythms of the heart, e.g., abnormal rhythms caused by damage to the conductive tissue that carries electrical signals, or abnormal rhythms caused by electrolyte imbalances. In a myocardial infarction (MI), the ECG can identify whether the heart muscle has been damaged in specific areas.

In some clinical settings, ECG monitoring is used for relatively short durations of time (e.g., minutes). However, certain patient conditions necessitate monitoring for prolonged periods of time (e.g., hours, days, weeks, months). Prolonged ECG monitoring can cause patient discomfort, as the adhesive material used to affix the electrodes to the skin can result in irritation. Furthermore, some patients suffer from allergies or sensitivities to adhesives (e.g., irritant contact dermatitis, allergic contact dermatitis), leading to rashes, swelling, blistering, and/or erythema. Additionally, patients with underlying dermatological conditions (including but not limited to patients with burns) cannot readily tolerate standard methods of ECG electrode placement, which requires skin abrasion prior to application of adhesive material. This situation is further complicated when ambulatory use is necessary; for example, when a dermatologically sensitive patient needing continual ECG monitoring must maintain mobility (e.g., while engaging in physical therapy or engaging in movement to prevent formation of blood clots). Without the use of adhesive material, ECG electrodes are prone to slippage across the surface of the skin, causing artifacts and rendering the monitoring procedure incompatible with telemetry. Adhesive-free suction ECG electrodes are available in which a bulb attached to a cup bearing the electrode is compressed to provide sufficient suction to affix the electrode to the patient's skin; however, their bulk and the level of negative pressure that such devices place on the skin render them uncomfortable and unsuitable for long-term or ambulatory use.

There is need in the art for improved devices for ECG electrode placement without use of adhesive material, and particularly for devices that are compatible with ambulatory monitoring (e.g., telemetry).

### SUMMARY OF THE INVENTION

The invention relates to devices and methods for monitoring cardiac function. Specifically, the invention relates to devices for secure placement of electrocardiogram (ECG) sensors (e.g., patches, electrodes) on the body of a subject.

ECG monitoring provides valuable data on cardiac function, such data being necessary to diagnose, monitor, and treat patients with a variety of conditions (including but not limited to patients with coronary artery disease, cardiac arrhythmia, myocardial infarction, stroke, heart transplant, lung transplant, syncope, heart failure, congestive heart failure, angina, or who have experienced medical treatments for cardiac conditions (e.g., stent placement, pacemaker placement, defibrillator placement, angioplasty, bypass surgery, catheterization) or who are taking medications or who have ingested other substances that may affect cardiac rhythm (e.g., caffeine, cold medications, cough medications, appetite suppressants, beta blockers, nicotine, thyroid medications, asthma medications, narcotics (e.g., cocaine), stimulants (e.g., amphetamines)). ECG is also used extensively for subjects without known pathological states, for example when monitoring and improving athlete performance. In general, ECG procedures require contact of the patient's skin with electrodes at various places on the body. Since slippage of the electrode across the skin surface results in significant artifact, standard ECG patch placement procedures require the skin to be prepared by shaving and abrasion, along with the use of strong adhesive material to keep the electrode in place. Such standard methods can cause serious discomfort for many patients, and some dermatologically-sensitive patients (e.g., patients with contact dermatitis; irritant contact dermatitis; allergic contact dermatitis; burns; blisters; rashes; eczema) are unable to tolerate the skin abrasion, the exposure to adhesives, or both. Additionally, even patients that can tolerate standard adhesive ECG patch placement for short periods of time (e.g., minutes, hours) can develop discomfort during longer periods of monitoring (e.g., days, weeks, months, years). The present invention provides devices and methods for adhesive-free placement of ECG electrodes. Such devices and methods are suitable for dermatologically-sensitive patients, and furthermore permit monitoring during patient movement (e.g., ambulation, physical therapy, athletic performance). Such devices and methods are also compatible with both short-term (e.g., minutes, hours) and long-term (e.g., days, weeks, years) monitoring, as well as intermittent monitoring.

The present invention provides devices (e.g., garments) for placement of ECG electrodes. The present invention is not limited to particular types of garments. In some embodiments, the present invention provides garments to be worn on the chest or torso of a subject (e.g., a halter, a brassiere, a tank top, a shirt, a vest, a sleeveless shirt, a shirt with partial sleeves, a shirt with full-length sleeves, a tube top, or any manner of garment that fully or completely encompasses the thoracic region of a subject). In some embodiments, garments of the present invention or a portion thereof (e.g., region(s) of the garment comprising aperture(s) for electrode insertion) wrap or cross the thoracic region of a subject on a bias or diagonal with the aim of facilitating close contact between the skin of a subject and insertable electrodes (Cho et al. (2009) J. Med. Syst. DOI: 10.1007/s10916-009-9356-8; herein incorporated by reference in its entirety).

The present invention is not limited by materials used for construction of embodiments of the invention. In some embodiments, the device is manufactured out of cloth textile(s). Textiles used for construction may be made from natural materials (e.g., wool, silk, cotton, jute, linen, hemp, bamboo, flax), synthetic materials (e.g., polyester, acetate, acrylic, nylon, spandex, olefin fiber, polylactide fiber, milk fiber, casein fiber), or a mixture of natural and synthetic materials. Textiles are not limited by the nature of thread count, warp, weave, weight, or other characteristics. In some embodiments, apertures for electrode insertion are positioned along a strip of material (e.g., fabric). In some embodiments, a strip of aperture-containing material is capable of unidirectional extension. In some embodiments, Veltex® brand fabric is used for a strip of aperature-containing material. In some embodiments, non-woven polymers or composites are used for construction (e.g., rubber, silicone, neoprene). In some embodiments, more than one material is used for construction.

In some embodiments, the present invention provides devices for placement of ECG sensors on the body of a patient, wherein the devices lack integration of said sensors in the device itself. In some embodiments, the present invention provides devices for placement of ECG sensors on the body of a patient, said devices lacking integrated conductors, wires, or transmitters. In some embodiments, the present invention provides devices for placement of ECG sensors on the body of a patient wherein said devices do not require specialized equipment, software, or hardware for interface with existing (e.g., standard) hospital ECG equipment and/or telemetry systems. In some embodiments, devices of the present invention are compatible with existing (e.g., standard) ECG electrodes, wires, computer hardware, and software programs. In some embodiments, the present invention provides a device constructed of material(s) that are worn comfortably by the patient and that therefore can be utilized for extended periods of time (e.g., days, weeks, months, years). In some embodiments, devices of the present invention find use with patients that have allergies or sensitivities to adhesives, or that have skin condition(s) that are incompatible with the typical electrode patch (e.g., irritant contact dermatitis, allergic contact dermatitis, burns, blisters).

In some embodiments, devices of the present invention are compatible with ECG electrode patches, said electrode patches lacking adhesive component(s). In some embodiments, electrode pole(s) is/are inserted through an aperture in the device, e.g., through a 3/16-inch hole between the one-inch material and the garment, followed by clamping the lead to the pole. In some embodiments, conductive material (e.g., conductive gel) is used to facilitate contact between the electrode and the patient's skin. Devices of the present invention are not limited by the position of the aperture(s), the number of apertures, the shape of the aperture(s), or the dimensions of the aperture(s). There may be one aperture or more than one aperture. In preferred embodiments, the device comprises a plurality of apertures. When more than one aperture is present, spacing between apertures may be less than 0.5 in, 0.5-1.0 in, 1.0-1.5 in, 1.5-2.0 in, 2.0-2.5 in, 2.5-3 in, 3-4 in, 4-5 in, 5-6 in, 6-10 in, 10 in or more. The distance between apertures may be constant or may vary at different positions within the garment. When a plurality of apertures is present, the apertures may be positioned in straight lines relative to each other or in non-linear arrangement. There may be 1, 2, 3, 4, 5, 6, 7, 8, 9, 10, 11, 12, 12-15, 15-20, 20-25, 25 or more apertures within the garment. The shape of the aperture(s) may be circular, square, triangular, rectangular, diamond, oval, irregular, or any other manner of shape. In some embodiments, the aperture is circular. When a plurality of apertures is present, the apertures may have the same shape or the shape may differ. The diameter of the aperture(s) may be less than 0.1 in, 0.1-0.15 in, 0.15-0.2 in, 0.2-0.25 in, 0.25-0.3 in, 0.3-0.4 in, 0.4-0.5 in, 0.5-1.0 in, 1.0-1.5 in, 1.5-2 in, 2 in or more.

In some embodiments, inserting an electrode through an aperture secures the electrode and restricts it from moving. Once the electrode(s) and lead(s) are positioned in the desired locations, the garment is placed on the patient's body. In some embodiments, a telemetry test is conducted to ascertain whether sufficient skin contact is occurring. In some embodiments, if one or more of the poles or the conductive gel does not have sufficient contact with the patient's skin resulting in artifactual telemetry readings, location-specific pressure adjustments may be made by inserting a removable electrode-compressive object behind the electrode. The electrode-compressive object may be a pillow, cushion, sphere, patch, cylinder, weight, netting, fabric, or other manner of insertable material that serves to provide localized pressure to the electrode. The electrode-compressive object may be in direct contact with the electrode, or may be in indirect contact (e.g., there may be fabric, or other material between the electrode-compressive object and the electrode; in one non-limiting example, a fabric pocket or flap may serve to hold the electrode-compressive object in place). In some embodiments, the electrode-compressive object is placed between the garment and the lead that is clamped to the pole, resulting in additional location-specific pressure being applied to the electrode (and/or conductive gel) and the skin to reduce skin slippage and artifactual readings. In some embodiments, the application of electrode-compressive object(s) is not required. Where a plurality of electrodes is used, an electrode-compressive object may be used with none of the electrodes, one of the electrodes, some of electrodes, or all of the electrodes. In some embodiments, devices of the present invention facilitate patient ambulation or mobility, e.g., allowing a patient to sit, stand, lie down, walk and move their arms with the same monitoring results as regular ECG patches with adhesive. In some embodiments, devices of the present invention correlate with telemetry artifact levels that are no different than occurring with standard (e.g., adhesive-containing) ECG patches. Certain embodiments of the invention find use with one ECG lead or more than one ECG lead (e.g., 1, 2, 3, 4, 5, 6, 7, 8, 9, 10, 11, 12, 13 or more leads). Certain embodiments of the invention find use with bipolar ECG leads. Certain embodiments of the invention find use with unipolar ECG leads. Embodiments of the invention are compatible with all manner of ECG electrodes (e.g., nickel-plate electrodes; Ag/AgCl electrodes; solid gel electrodes; carbon snap electrodes; liquid gel electrodes). In preferred embodiments, no adhesive material is present in or near the electrode component.

Methods of monitoring ECG data using embodiments of the present invention are not limited by the duration of use. Embodiments of the present invention may be used for less than 60 minutes; 1-6 h; 6-12 h; 12-24 h; 1-5 days; 5-10 days; 10-30 days; 30 days or more: 1 year or more. Embodiments of the present invention may be used for intermittent monitoring, e.g., monitoring for a short (e.g., minutes, hours) or long (e.g., days, weeks, months, years) period of time followed by a period of no monitoring, then followed by a resumption of monitoring for a short or long duration of time.

Embodiments of the present invention are compatible with patient movement, without limitation to the type of patient movement. Movement may include but is not limited to ambulation; exercise (e.g., athletic movement, whether for training or competition purposes; general purpose fitness movement); physical therapy; occupational therapy; and activities of daily life (e.g., movements associated with dressing, personal hygiene, eating, food preparation, work, and social interaction).

Embodiments of the present invention are compatible with telemetry systems, without limitation to the nature of such systems, the components therein, or the frequency at which they operate.

In certain embodiments, the present invention provides a device for placement of at least one ECG electrode on the body of a subject, comprising a garment, at least one aperture, and at least one electrode-compressive object. In some embodiments, the aperture is capable of accommodating an electrode inserted therethrough. In some embodiments, a plurality of apertures is present. In some embodiments, the aperture occurs within material capable of unidirectional elongation. In some embodiments, the material is Veltex® fabric. In some embodiments, the electrode-compressive object is selected from object such as a cushion, a pillow, a pad, a roll of textile material, a cylinder, a rod, and a ball. In some embodiments, the garment encompasses the torso of a subject. In some embodiments, the garment is a type such as a halter, a brassiere, a tank top, a shirt, a vest, a sleeveless shirt, a shirt with partial sleeves, a shirt with full-length sleeves, and a tube top. In some embodiments, the garment provides cross-body construction. In some embodiments, the electrode-compressive object is inserted behind material comprising an aperture. In some embodiments, an electrode is inserted through the aperture.

In certain embodiments, the present invention provides a method of capturing electrocardiogram data using devices described herein 1.

Additional embodiments will be apparent to persons skilled in the relevant art based on the teachings contained herein.

### DESCRIPTION OF THE DRAWINGS

FIG. 1 shows an embodiment of the invention, further described in Example 3 infra.

### DEFINITIONS

To facilitate an understanding of the present invention, a number of terms and phrases are defined below:

As used herein, the term “electrocardiogram” or “ECG” refers to a procedure in which electrical activity of the heart is detected using electrode(s) placed on the skin of a subject.

As used herein, the term “electrode” refers to an electric conductor through which an electric current enters or leaves an electrolytic cell or other medium. In electrocardiogram methods and systems, electrode(s) are used to detect electrical activity of the heart and facilitate the transmission of waveform data for visual display.

As used herein, the terms “subject” and “patient” refer to any animal, such as a mammal like a dog, cat, bird, livestock, and preferably a human (e.g. a human with a cardiovascular condition such as cardiovascular disease, angina, or cardiac arrhythmia).

As used herein, the term “aperture” refers to an opening in a surface. In some embodiments, apertures permit insertion of another component (e.g., an ECG electrode) such that the component is partially or totally surrounded by the aperture-bearing surface.

As used herein, the term “electrode-compressive object” refers to an a component that is placed behind an electrode that is inserted through an aperture of a garment embodiment of the present invention, pressing the electrode more firmly towards the skin of the garment wearer than would occur in absence of the electrode-compressive object, without limitation to the dimensions of the electrode-compressive object or the material used for its construction. In some embodiments, the electrode-compressive object is a pillow, pad, cushion, or other three-dimensional pliable object. In some embodiments, the electrode-compressive object is a rod, box, cylinder, or other three-dimensional non-pliable (e.g., non-yielding) object.

As used herein, the term “cross-body” or “cross-body construction” refers to the extension of material used to construct a garment embodiment of the present invention such that said extension occurs in a direction other than perpendicular to the long axis of the subject's body. In some embodiments, cross-body construction is provided by directing the warp of a woven textile in a diagonal direction relative to the long axis of the subject's body when a garment is worn by a subject. In some embodiments, cross-body construction is provided by directing the weft of a woven textile in a diagonal direction relative to the long axis of the subject's body when a garment is worn by a subject. In some embodiments, cross-body construction is provided by using non-woven material for construction of a garment, wherein said non-woven material is capable of unidirectional extension, and wherein said unidirectional extension occurs at a diagonal relative to the long axis of the subject's body.

### DETAILED DESCRIPTION OF THE INVENTION

The invention relates to devices and methods for monitoring cardiac function. Specifically, the invention relates to devices for secure placement of electrocardiogram (ECG) sensors (patches, electrodes) on the body of a subject. In preferred embodiments, said placement occurs without the use of adhesive material. Therefore, some embodiments of the present invention find use with patients having sensitivities to or intolerance of adhesive compounds (e.g., due to irritant contact dermatitis, due to allergic contact dermatitis).

Various devices known in the art such as chest belts or harnesses, gloves and sleeves, shirts or jackets etc. operate with the use of integrated ECG sensors or textile electrodes for electrocardiogram (ECG) monitoring (U.S. Pat. No. 6,006,125; U.S. Pat. No. 7,474,910; U.S. Pat. No. 7,308,294; U.S. patent application Ser. No. 11/749,248; U.S. patent application Ser. No. 11/749,253; U.S. Pat. No. 7,173,437; U.S. Pat. No. 6,842,722; each herein incorporated by reference in its entirety). The integration of sensors or electrodes in all of these devices causes significant problems in clinical settings, one of the most serious drawbacks being slippage across the patient's skin, resulting in artifactual readings. Attempts to prevent such slippage include providing manual pressure to the sensor or textile, which hinders freedom of movement; or application of pressure by construction of the device with compressive material such as Neoprene®, which itself can cause skin irritation if remaining in contact with the skin for prolonged periods. Additionally, such integrated sensor devices are not directly compatible with hospital telemetry, and often require secondary programs or equipment for continuous patient monitoring. Lack of direct compatibility increases both cost and complexity of the system, leading to reluctance of medical staff to utilize integrated sensor devices and systems for fear of compromising patient safety.

In some embodiments, the present invention provides devices for placement of ECG sensors on the body of a patient, wherein the devices lack integration of said sensors in the device itself. In some embodiments, the present invention provides devices for placement of ECG sensors on the body of a patient, said devices lacking integrated conductors, wires, or transmitters. In some embodiments, the present invention provides devices for placement of ECG sensors on the body of a patient wherein said devices do not require specialized equipment, software, or hardware for interface with existing (e.g., standard) hospital ECG equipment and/or telemetry systems. In some embodiments, devices of the present invention are compatible with existing (e.g., standard) ECG electrodes, wires, computer hardware, and software programs. In some embodiments, the present invention provides a device constructed of material(s) that are worn comfortably by the patient and that therefore can be utilized for extended periods of time (e.g., days, weeks, months, years). In some embodiments, devices of the present invention find use with patients that have allergies or sensitivities to adhesives, or that have skin condition(s) that are incompatible with the typical electrode patch (e.g., irritant contact dermatitis, allergic contact dermatitis, burns, blisters).

In some embodiments, devices of the present invention are compatible with ECG electrode patches, said electrode patches lacking adhesive component(s). In some embodiments, electrode pole(s) is/are inserted through an aperture in the device, e.g., through a 3/16-inch hole between the one-inch material and the garment, followed by clamping the lead to the pole. In some embodiments, conductive material (e.g., conductive gel) is used to facilitate contact between the electrode and the patient's skin. Devices of the present invention are not limited by the position of the aperture(s), the number of apertures, the shape of the aperture(s), or the dimensions of the aperture(s). There may be one aperture or more than one aperture. In preferred embodiments, the device comprises a plurality of apertures. When more than one aperture is present, spacing between apertures may be less than 0.5 in, 0.5-1.0 in, 1.0-1.5 in, 1.5-2.0 in, 2.0-2.5 in, 2.5-3 in, 3-4 in, 4-5 in, 5-6 in, 6-10 in, 10 in or more. The distance between apertures may be constant or may vary at different positions within the garment. When a plurality of apertures is present, the apertures may be positioned in straight lines relative to each other or in non-linear arrangement. There may be 1, 2, 3, 4, 5, 6, 7, 8, 9, 10, 11, 12, 12-15, 15-20, 20-25, 25 or more apertures within the garment. The shape of the aperture(s) may be circular, square, triangular, rectangular, diamond, oval, irregular, or any other manner of shape. In some embodiments, the aperture is circular. When a plurality of apertures is present, the apertures may have the same shape or the shape may differ. The diameter of the aperture(s) may be less than 0.1 in, 0.1-0.15 in, 0.15-0.2 in, 0.2-0.25 in, 0.25-0.3 in, 0.3-0.4 in, 0.4-0.5 in, 0.5-1.0 in, 1.0-1.5 in, 1.5-2 in, 2 in or more.

In some embodiments, inserting an electrode through an aperture secures the electrode and restricts it from moving. Once the electrode(s) and lead(s) are positioned in the desired locations, the garment is placed on the patient's body. In some embodiments, a telemetry test is conducted to ascertain whether sufficient skin contact is occurring. In some embodiments, if one or more of the poles or the conductive gel does not have sufficient contact with the patient's skin resulting in artifactual telemetry readings, location-specific pressure adjustments may be made by inserting a removable electrode-compressive object behind the electrode. The electrode-compressive object may be a pillow, cushion, sphere, patch, cylinder, weight, netting, fabric, or other manner of insertable material that serves to provide localized pressure to the electrode. The electrode-compressive object may be in direct contact with the electrode, or may be in indirect contact (e.g., there may be fabric, or other material between the electrode-compressive object and the electrode; in one non-limiting example, a fabric pocket or flap may serve to hold the electrode-compressive object in place). In some embodiments, the electrode-compressive object is placed between the garment and the lead that is clamped to the pole, resulting in additional location-specific pressure being applied to the electrode (and/or conductive gel) and the skin to reduce skin slippage and artifactual readings. In some embodiments, the application of electrode-compressive object(s) is/are not required. Where a plurality of electrodes is used, an electrode-compressive object may be used with none of the electrodes, one of the electrodes, some of electrodes, or all of the electrodes. In some embodiments, devices of the present invention facilitate patient ambulation or mobility, e.g., allowing a patient to sit, stand, lie down, walk and move their arms with the same monitoring results as regular ECG patches with adhesive. In some embodiments, devices of the present invention correlate with telemetry artifact levels that are no different than occurring with standard (e.g., adhesive-containing) ECG patches.

### Therapeutic Indications

Some embodiments of the present invention find use with patients that cannot tolerate ordinary ECG patches because they are allergic to adhesives, and/or have adhesive- and abrasion-incompatible skin conditions. Examples of such skin conditions include but are not limited to psoriasis, eczema, dermatitis (e.g., irritant contact dermatitis, allergic contact dermatitis), rashes, blisters, and burns. Embodiments of the present invention find use with any patient for whom ECG monitoring is desired. Some embodiments find use with patients having chest pain or angina; with patients who have experienced or who are at risk for experiencing syncope episodes (e.g., vasovagal syncope, neurocardiogenic syncope); with patients who have experienced heart or lung surgery; with patients who have experienced a medical procedure that places them at risk for cardiac arrhythmia (e.g., cardiac catheterization, angioplasty, stent placement); with patients who are known to have or suspected to have heart or lung disease; with patients who are known to have or suspected to have cardiac arrhythmia; with patients who have received or who are candidates to receive internal cardiac devices (e.g., pacemakers, artificial internal cardiac defibrillators, pacemaker/defibrillator devices); or with patients who are taking medications that cause or that have risk of causing cardiac arrhythmia(s).

### Settings for Use

Embodiments of the present invention find use in a variety of settings, including but not limited to hospitals, clinics, emergency transport (EMS), home use, nursing homes, fitness facilities, exercise physiology facilities (e.g., athlete training facilities), assisted living facilities, and in the field (e.g., on battlefields, in military medical treatment facilities, at sporting events, during outdoor recreational events, during search-and-rescue operations). In some embodiments, devices of the present invention may be provided as durable medical equipment in clinical settings (e.g., in hospitals, in clinics, during therapy sessions, during medical appointments). Once provided for patient use, in some embodiments, devices of the present invention may be utilized multiple times in the same or different settings (e.g., provided for use in hospital, and later utilized for patient monitoring in a home setting, or vice versa). In some embodiments, devices of the present invention are disposable. In some embodiments, devices of the present invention may be provided to emergency transport vehicles. Such availability for emergency transport finds particular use when medical staff encounter patients with dermatological sensitivity to or incompatibility with standard adhesive-containing ECG patches, or for whom dermatological status is unknown (e.g., where a patient is unconscious and status of allergies or sensitivities to adhesive is unknown). Such devices also find use whenever customized electrode positioning and/or customized placement of electrode-compressive objects is desired. Such devices may be provided directly to patients for self-monitoring, particularly when a patient desires to avoid the use of adhesive and/or skin abrasion for electrode placement, and/or when a patient desires customized electrode positioning and/or customized placement of electrode-compressive objects. In some embodiments, devices of the present invention find use with patients of a variety of ages and health status (e.g., pediatric patients, adult patients, geriatric patients, disabled patients, pregnant patients, infant or neonatal patients). In some embodiments, devices of the present invention find use in research settings. In some embodiments, devices of the present invention find use for non-human subjects, e.g., for veterinary applications, for livestock performance (e.g., with racehorses), or with research animals.

The present invention provides kits for electrode positioning on the body of a subject. In some embodiments, kits comprise components such as a garment embodiment of the present invention and/or at least one electrode-compressive object. Kits may include additional components such as adhesive-free electrode patches, conductive gel, and leads.

### Electrocardiography

The present invention provides devices for use with electrocardiography (ECG) without limitation to specific ECG regime, procedure, number of leads, number of electrodes, duration of monitoring, or nature of ECG data captured. In standard ECG protocols, ten electrodes are used for a 12-lead ECG. Locations for placement of limb electrodes are well-known in the art (Peberdy et al. (1993) Am. J. Emer. Med. 11:403-405; Table 1). In standard protocols, limb electrodes can be far down on the limbs or close to the hips/shoulders, but they generally must be even (left vs right).

TABLE 1
Electrode placement for standard 12-lead ECG protocols.

| Electrode | |
|---|---|
| label | |
| (in the | |
| USA) | Electrode placement |
| RA | On the right arm, avoiding bony prominences. |
| LA | In the same location that RA was placed, but on the |
| | left arm this time. |
| RL | On the right leg, avoiding bony prominences. |
| LL | In the same location that RL was placed, but on the |
| | left leg this time. |
| V1 | In the fourth intercostal space (between ribs 4 & 5) just |
| | to the right of the sternum (breastbone). |
| V2 | In the fourth intercostal space (between ribs 4 & 5) just |
| | to the left of the sternum. |
| V3 | Between leads V2 and V4. |
| V4 | In the fifth intercostal space (between ribs 5 & 6) in the |
| | mid-clavicular line (the imaginary line that extends down |
| | from the midpoint of the clavicle (collarbone). |
| V5 | Horizontally even with V4, but in the anterior axillary |
| | line. (The anterior axillary line is the imaginary line |
| | that runs down from the point midway between the middle |
| | of the clavicle and the lateral end of the clavicle; the |
| | lateral end of the collarbone is the end closer to |
| | the arm.) |
| V6 | Horizontally even with V4 and V5 in the midaxillary |
| | line. (The midaxillary line is the imaginary line that extends |
| | down from the middle of the patient's armpit.) |

### Telemetry

Biotelemetry (telemetry, medical telemetry) involves the application of telemetry in the medical field to remotely monitor various vital signs of ambulatory patients. The most common usage for biotelemetry is in dedicated cardiac care telemetry units or step-down units in hospitals. A typical biotelemetry system comprises sensors appropriate for the particular signals to be monitored; battery-powered transmitters worn by patients; a radio antenna and receiver; and a display unit capable of concurrently presenting information from multiple patients. Typically, telepacks or transmitters wirelessly send data to an antenna network which in turn sends the data to a central station monitor. The central station then displays the ECG waveforms of the patients on telemetry and issues alarms to inform staff about clinically significant events. In the traditional sense, each transmitter sends its data out on a different frequency so that the transmitters don't interfere with each other. Because of crowding of the radio spectrum due to the recent introduction of HDTV in the United States and many other countries, the FCC as well as similar agencies elsewhere have recently begun to allocate dedicated frequency bands for exclusive biotelemetry usage, for example, the Wireless Medical Telemetry Service (WMTS). The FCC has designated the American Society for Healthcare Engineering of the American Hospital Association (ASHE/AHA) as the frequency coordinator for the Wireless Medical Telemetry Service (WMTS). In addition, there are many biotelemetry products that utilize commonly available standard radio devices such as Bluetooth and IEEE 802.11.

In some embodiments, when devices of the present invention are used with telemetry systems, telemetry can still indicate changes in heart rhythm or rate and alarms can be set off on the basis of such changes. As occurs when using standard adhesive ECG patches, such alarms can indicate changes in events unrelated to the patient health. Such health-unrelated events include but are not limited to patient movement (e.g., due to arm movement causing movement of leads, as occurs with brushing one's teeth); pressure or tapping on leads; disconnected leads; low battery; or patient ambulation outside of battery range.

### Types of Garments

The present invention is not limited to particular types of garments. In some embodiments, the present invention provides garments to be worn on the chest or torso of a subject (e.g., a halter, a brassiere, a tank top, a shirt, a vest, a sleeveless shirt, a shirt with partial sleeves, a shirt with full-length sleeves, a tube top, or any manner of garment that fully or completely encompasses the thoracic region of a subject). In some embodiments, garments of the present invention or a portion thereof (e.g., region(s) of the garment comprising aperture(s) for electrode insertion) wrap or cross the thoracic region of a subject on a bias or diagonal with the aim of facilitating close contact between the skin of a subject and insertable electrodes (Cho et al. (2009) J. Med. Syst. DOI: 10.1007/s10916-009-9356-8; herein incorporated by reference in its entirety).

### Materials Used for Construction

Embodiments of the present invention are not limited by materials used for their construction. In some embodiments, the device is manufactured out of cloth textile(s). Textiles used for construction may be made from natural materials (e.g., wool, silk, cotton, jute, linen, hemp, bamboo, flax), synthetic materials (e.g., polyester, acrylic, nylon, spandex, olefin fiber, polylactide fiber, milk fiber, casein fiber), composites thereof, or a mixture of natural and synthetic materials, or composites thereof. Textiles are not limited by the nature of thread count, warp, weave, weight, or other characteristics. In some embodiments, apertures for electrode insertion are positioned along a strip of material (e.g., fabric). In some embodiments, a strip of aperture-containing material is capable of unidirectional extension. In some embodiments, Veltex® brand fabric is used for a strip of aperature-containing material. In some embodiments, non-woven polymers or composites are used for construction (e.g., rubber, silicone, neoprene, elastic polymers or composites, Velcro® material). In some embodiments, more than one material is used for construction.

### Contact Dermatitis

Some embodiments of the present invention find use with patients having dermatitis (e.g., irritant contact dermatitis, allergic contact dermatitis). Contact dermatitis (CD) is acute inflammation of the skin caused by irritants or allergens. The primary symptom is pruritus. Skin changes range from erythema to blistering and ulceration. Diagnosis is by exposure history, examination, and sometimes skin patch testing. Treatment entails antipruritics, topical corticosteroids, and avoidance of causes.

Irritant contact dermatitis (ICD) accounts for 80% of all cases of CD. It is a nonspecific inflammatory reaction to substances contacting the skin during which the immune system is not activated. ICD is more painful than pruritic. Signs range from mild erythema to hemorrhage, crusting, erosion, pustules, bullae, and edema. Numerous substances may trigger ICD, including substances present in adhesive materials. Environmental and patient factors may influence the development and/or course of ICD events. Properties of the irritant (e.g., extreme pH, solubility in the lipid film on skin), environment (e.g., low humidity, high temperature, high friction), and patient (e.g., very young or old) influence the likelihood of developing ICD. ICD is more common among atopic patients, in whom ICD also may initiate immunologic sensitization and hence allergic CD.

Allergic contact dermatitis (ACD) is a type IV cell-mediated hypersensitivity reaction that has 2 phases: 1) sensitization to an antigen, and 2) allergic response after reexposure. In the sensitization phase, allergens are captured by Langerhans' cells (dendritic epidermal cells), which migrate to regional lymph nodes where they process and present the antigen to T cells. The process may be brief (e.g., 6 to 10 days for strong sensitizers) or prolonged (years for weak sensitizers). Sensitized T cells then migrate back to the epidermis and activate on any reexposure to the allergen, releasing cytokines, recruiting inflammatory cells, and leading to the characteristic symptoms and signs of ACD.

In autoeczematization, epidermal T cells activated by an allergen migrate locally or through the circulation to cause dermatitis at sites remote from the initial trigger. However, contact with fluid from vesicles or blisters cannot trigger a reaction elsewhere on the patient or on another person.

Multiple allergens cause ACD and cross-sensitization among agents is common. When cross-sensitization occurs, exposure to one substance can result in an allergic response after exposure to a different but related substance. Agents commonly present in adhesive materials that can cause ACD include but are not limited to including acrylic monomers, epoxy compounds, vat dyes, rubber accelerators, and formaldehyde. Case reports of ACD resulting from exposure to adhesive bandage materials have been reported (Norris et al. (1990) Dermatol. Clin. 8:147-152; herein incorporated by reference in its entirety). Allergens reported in this study included tricresyl phosphate, the plasticizer in the vinyl backing; and 2,5-di(tertiary-amyl)hydroquinone, the antioxidant in the adhesive.

In ACD, the primary symptom is intense pruritus; pain is usually the result of excoriation or infection. Skin changes range from transient erythema through vesiculation to severe swelling with bullae, ulceration, or both. Changes often occur in a pattern, distribution, or combination that suggests a specific exposure, such as a shape matching that of an adhesive bandage. The dermatitis is typically limited to the site of contact but may later spread due to scratching and auto eczematization. In systemically induced ACD, skin changes may be distributed over the entire body.

Treatment of contact dermatitis, whether ICD or ACD, necessitates avoiding exposure to the trigger (e.g., irritant, allergen).

### Burns

In some embodiments, the present invention finds use for burn patients. Depending on the severity of burns, the epidermis of burn patients may be in poor condition and unable to tolerate the dermal abrasion and adhesives involved in standard ECG patch placement. Types of burns include thermal burns, chemical burns, and radiation burns. Thermal burns can be further classified according to skin depth and percentage of total body area burned.

Burn depth is described as superficial, partial thickness, or full thickness (corresponding to first, second, or third degree.

Superficial (first-degree) burns involve only the epidermis. Characteristics of first-degree burns include tissue blanching under pressure, erythematous tissue, minimal tissue damage, possible presence of edema, and generally the absence of blisters. These wounds are dry, red, painful, and generally heal in 3-6 days without scarring. Sunburn is a classic example of first-degree burn.

Partial-thickness burns (second-degree) are often further delineated into superficial and deep types. Epidermis and portions of the dermis are involved, and blisters usually form either very quickly or within 24 hours. Superficial and deep partial-thickness can be difficult to differentiate clinically. The difference lies in the depth of penetrance into the dermis with the transition occurring at about half of dermal depth. Superficial partial-thickness burns usually blanch and do not result in scarring. Deep partial-thickness burns often do not blanch and do scar. The deeper the injury, the longer the healing time, which may vary from 7-21 days in the more superficial dermis burns to greater than 21 days in the deep dermis burns. Adnexal structures (eg, sweat glands, hair follicles) are often involved, but enough of these structures are preserved for function, and the epithelium lining them can proliferate and allow for re-growth of skin. If deep second-degree burns are not cared for properly, edema, which accompanies the injury, and decreased blood flow in the tissue can result in conversion to full-thickness burn. These wounds are red, wet, and painful (with decreasing pain, color, and moisture with increasing depth into the dermis).

Full-thickness (third-degree) burns extend completely through the skin to subcutaneous tissue. They may involve underlying structures including tendon, nerves, muscle, or bone (sometimes previously referred to as fourth-degree burn). These burns are characterized by charring of skin or a translucent white color, with coagulated vessels visible below. The area is insensate, but the patient complains of pain, which is usually a result of surrounding second-degree burn. As all of the skin tissue and structures are destroyed, healing is very slow. Full-thickness burns are often associated with extensive scarring because epithelial cells from the skin appendages are not present to repopulate the area. These wounds vary from waxy white, to charred and black often with a leathery texture, they are dry and usually painless to touch. These wounds generally do not heal spontaneously.

Burn extent is expressed in terms of body surface area involvement. The more body surface area (BSA) involved in a burn, the greater the morbidity and mortality rates and the difficulty in management. An individual's palmar surface classically represents 1% of the BSA, but, in actuality, it represents about 0.4%, whereas the entire hand represents about 0.8%. A simple method to estimate burn extent is to use the patient's palmar surface including fingers to measure the burned area. Burn extent is calculated only on individuals with partial-thickness or full-thickness burn. Methods of estimating the extent of burn injury include but are not limited to the Rule of Nines and the Lund and Broder Burn Chart.

### EXAMPLES

The following examples are provided in order to demonstrate and further illustrate certain preferred embodiments and aspects of the present invention and are not to be construed as limiting the scope thereof.

### Example 1

An embodiment was constructed using a soft piece of textile material fashioned into approximately 2-inch wide strips. The length of the first strip was adequate to encompass the chest of a female patient below the breasts. Velcro was attached to the ends to make the first strip to make it adjustable in size. Second and third strips were attached to the first strip to form loops at a 90 degree angle to the first strip, each loop positioned over one shoulder of the patient. A fourth strip went across the front from one shoulder strip to the other and just above the breasts, running parallel to the first strip. Apertures were placed in strategic locations for insertion of electrodes. Adhesive-free ECG patches were placed between two pieces of material. The device was then tested and it was found that the telemetry was poor and unsatisfactory, rendering the device unsuitable for use.

### Example 2

A second embodiment was constructed with the aim of decreasing skin slippage and artifactual readings observed in the first embodiment (Example 1). An aperture corresponding to the shape and size of a standard ECG patch was cut out from a piece of material. A hole in the center of the material was cut out for the purpose of accommodating conductive gel. Standard ECG patches, including electrodes, were placed in various locations between a woman's brassiere and the subject's skin and held stationary by tightening the straps of the brassiere. Patch placement locations were under each shoulder strap on the front (chest/shoulder region); three patches across the region underneath the breasts on the chest; and one patch between the breasts on the chest. Telemetry tests resulted in moderate success with data collection; however, any subject movement resulted in substantial artifact, and in some cases subject movement resulted in disruption of the contact between the conductive gel/electrode and the skin.

A modification was made in which the fabric patches were pinned in place to the brassiere. This modification resulted in improved telemetry data collection. However, the patch between the breasts continued to slip. Therefore, a rolled-up piece of thicker fabric (terrycloth washcloth) was placed behind this patch to provide additional pressure. This further improved the quality of telemetry data collection; however, subject movement still resulted in electrode slippage and artifactual data.

### Example 3

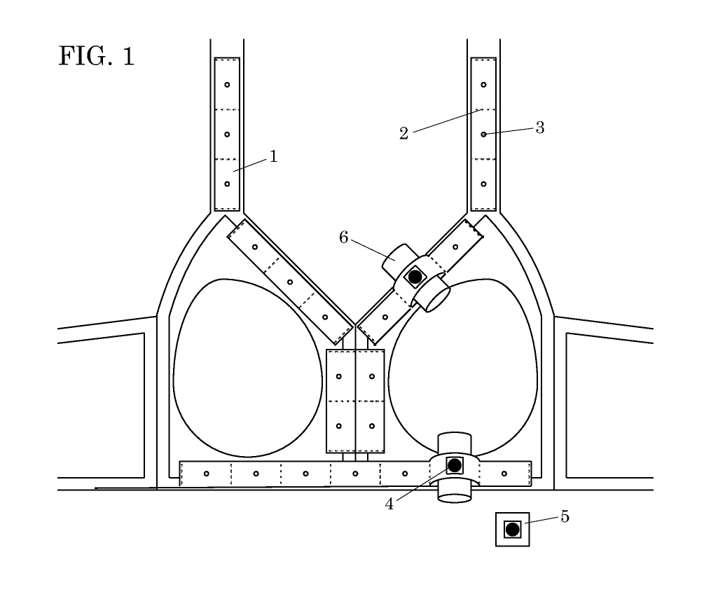

A third embodiment is shown in FIG. 1. This apparatus is in the shape of a women's brassiere; alternative embodiments can be shaped and sized for men, women or children. Material (Veltex® brand fabric) was cut into 1 in.-wide strips. Each strip was sewn down in strategic locations at 2-inch intervals with a 3/16-inch hole in the middle of each two-inch interval. The strips were sewn in the front of the brassiere, down along each strap, crossing between each breast, and below each breast. The crossing of the strips gave stability to the apparatus and better conductivity with the electrode gel and the skin.

Specifically, referring to FIG. 1, strips of fabric (1) capable of unidirectional expansion (Veltex® brand fabric) were affixed to a garment (Playtex® 18-hour brassiere) by stitches (2) sewn at regular intervals, the stitches being perpendicular to the long axis of the fabric strips, thereby creating loops of Veltex fabric. Circular apertures (3) were present within each segment of Veltex fabric. ECG electrodes (4) were placed by inserting the electrode at desired aperture position(s) such that the surface of the electrode emerged through the aperture, directed towards the skin of the garment wearer. Electrode-compressive object(s) (6) were placed between the electrode and the garment to provide pressure on the electrode against the patient's skin, reducing slippage. Before initiation of ECG monitoring, conductive gel membrane (5) was affixed on the face of the electrode, between the patient's skin and the electrode.

Tests were conducted with the help of medical staff (registered nurses specialized in the care of cardiac patients and the use of hospital telemetry). The medical staff found that the embodiment worked sufficiently for monitoring a patient's heart rhythm. The apparatus enabled telemetry with a plurality of electrodes, from which six precordial chest leads V1-V6 and up to 12 leads were selectively located, depending upon the chest size of the individual, without the use of adhesives on the electrodes. Each electrode was stabilized to suppress motion artifact. The cross-chest arrangement of the Veltex® strips resulted in electrode position stabilization. The telemetry read-out of ECG signal for the apparatus was the higher quality in comparison to embodiments not incorporating cross-chest construction (e.g., Example 1). Conductivity did not differ in comparison to standard procedures of applying the electrode patches directly to the skin with the regular use of telemetry.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in cardiac monitoring, electrophysiology, or related fields are intended to be within the scope of the following claims.

I claim:

1. A device for placement of at least one ECG electrode on the body of a subject, comprising a garment, at least one aperture, and at least one electrode-compressive object.

2. The device of claim 1, wherein said aperture is capable of accommodating an electrode inserted therethrough.

3. The device of claim 1, comprising a plurality of apertures.

4. The devise of claim 1, wherein said aperture occurs on material capable of unidirectional elongation.

5. The device of claim 4, wherein said material is Veltex® fabric.

6. The device of claim 1, wherein said electrode-compressive object is selected from the group consisting of a cushion, a pillow, a pad, a roll of textile material, a cylinder, a rod, and a ball.

7. The device of claim 1, wherein said garment encompasses the torso of a subject.

8. The device of claim 7, wherein said garment is selected from the group consisting of halter, a brassiere, a tank top, a shirt, a vest, a sleeveless shirt, a shirt with partial sleeves, a shirt with full-length sleeves, and a tube top.

9. The device of claim 1, wherein said garment provides cross-body construction.

10. The device of claim 1, wherein said electrode-compressive object is inserted behind material comprising an aperature.

11. The device of claim 10, wherein an electrode is inserted through said aperture.

12. A method of capturing electrocardiogram data using the device of claim 1.

13. An article of manufacture for placement of at least one ECG electrode on the body of a subject, comprising a garment, at least one aperture, and at least one electrode-compressive object.
